(19) United States
(12) Patent Application Publication
(10) Pub. No.: US 2013/0206138 A1
(43) Pub. Date: Aug. 15, 2013

(54) TRACHEAL TUBE WITH FLAT MOUTH AND SIDE OPENING AND GUIDE CORE

(71) Applicants: Zhiyang Chen, Shanghai (CN); Ruiguang Chen, Shanghai (CN); Jin Liu, Shanghai (CN)

(72) Inventors: Zhiyang Chen, Shanghai (CN); Ruiguang Chen, Shanghai (CN); Jin Liu, Shanghai (CN)

(21) Appl. No.: 13/578,612

(22) PCT Filed: Jan. 26, 2011
(86) PCT No.: PCT/CN2011/000123

§ 371 Date: Oct. 9, 2012

(30) Foreign Application Priority Data
Dec. 29, 2010 (CN) 201010620049

(51) Int. Cl. A61M 16/04

(52) U.S. Cl. CPC A61M 16/0488; A61M 16/0434
USPC 128/200.26

(57) ABSTRACT

A tracheal tube with a flat mouth and a side opening for trachea intubation is provided. The front end of the tracheal tube is a flat mouth with circular cross section. The tracheal tube has a side opening (2) and an air pocket (3). The inner diameter of the tracheal tube is 5 to 10 mm. A guide core for use with the tracheal tube is composed of a hemisphere, a front cylinder, a truncated cone and a rear cylinder. The hemisphere, front cylinder, truncated cone and rear cylinder of the guide core reach the trachea through glottis in turn during intubation, and then the tracheal tube reaches the trachea. Said tracheal tube and guide core avoid blocking the doctor' view, which greatly improving success ratio of intubation, and avoiding injury to the throat by the tracheal tube.

### 1. FIELD OF TECHNOLOGY

This invention involves an endotracheal tube used clinically on patients under anesthesia, for emergency care and intensive care. The front end has a flat opening. Its inner diameter is 5-10 mm. The endotracheal tube has a side opening and an air-pocket cuff. A guide core is used to guide the endotracheal tube into the trachea. The front end of the guide core is a hemisphere, its diameter being 3-5 mm. It is followed by a front cylinder, which is approximately 30-50 mm in length, its diameter being equal to the diameter of the leading hemisphere. This is followed by a truncated cone, the diameter of the front cross section of the truncated cone being equal to the diameter of the front cylinder. The diameter of the rear cross section is equal to the diameter of the following rear cylinder, and the truncated cone is 10-30 mm in length. The truncated cone is followed by the rear cylinder, which is 5-9 mm in diameter and 50-70 cm in length.

### 2. TECHNICAL BACKGROUND

Presently, an endotracheal tube with a slanted mouth at the front and a side opening is commonly used worldwide in clinical settings such as anesthesia. The slanted mouth at the front of an endotracheal tube is meant to adapt to the shape of the glottis (i.e., “1” shaped). This is to make it easier to slip the endotracheal tube through the glottis. However, because the slanted area at the front of the endotracheal tube is relatively large, it can easily get stuck at the glottis, epiglottis and larynx to make intubation more difficult. Damage to the throat by the slanted mouth can happen easily after repeated intubation attempts with brute force. This can lead to laryngeal edema, respiratory distress and even patient asphyxia. As a matter of fact, this is the most common cause of patient death encountered during intubation. When intubation becomes difficult, regardless of whether a bronchoscope or a bougie is used to assist intubation, after the successful insertion of a bronchoscope or bougie into the trachea, it is often difficult to push the endotracheal tube further down the windpipe. This is because the endotracheal tube can easily get stuck when its slanted mouth encounters the piriform sinus, epiglottis or vocal cords if the leading edge of the bronchoscope or bougie forms an angle with the insertion end of the endotracheal tube. When brute force is used to push the tube down the trachea, it can cause damage to the vocal cords, epiglottis and larynx. The bronchoscope may also be damaged. Intubation becomes very difficult. To overcome this problem, an endotracheal tube with a flat mouth, a side opening and a guide core was designed. The leading edge of the endotracheal tube has a circularly shaped flat mouth. The inner diameter at the opening is 5-10 mm. In addition, there is a side opening to enhance ventilation. The endotracheal tube is equipped with a guide core for entering the trachea. The front of the guide core is a hemisphere with a diameter of 3-5 mm. It is followed by a front cylinder, which is 30-50 mm in length and has adiameter equal to the diameter of the hemisphere. It is then followed by a truncated cone having a length of 10-30 mm. The truncated cone is followed by a rear cylinder, which is 5-9 mm in diameter and 50-70 cm in length. The advantage of this guide core is that its front end is hemispherical in shape, which makes it easier to pass through the glottis to minimize damage to the vocal cords and the larynx. The view of the physician performing intubation under the laryngoscope is not obstructed by the endotracheal tube. The leading hemisphere and the front cylinder of the guide core enter the glottis first, followed by the gradually thickened truncated cone and rear cylinder. This is followed by the insertion of the endotracheal tube, which has an inner diameter slightly larger than the diameter of the rear cylinder of the guide core. Regardless of whether intubation is routine or difficult, this endotracheal tube with a flat mouth, side opening and guide core can provide a clear view for the physician performing intubation and avoids damage to the vocal cords, epiglottis and piriform sinus in order to significantly improve the success rate of intubation.

### 3. SUMMARY OF INVENTION

Presently, an endotracheal tube with a slanted mouth at the front is commonly used clinically. Once it gets stuck when passing through the glottis, damage to the vocal cords, epiglottis and piriform sinus can occur quite easily. An endotracheal tube with a flat mouth, a side opening and a guide core was designed. The front end is a circularly shaped flat mouth. Its guide core passes through the glottis in the following order; i.e., front hemisphere, front cylinder, truncated cone and rear cylinder. The endotracheal tube is then inserted into the trachea along the guide core. Afterward, the guide core can be withdrawn. Damage to the larynx can be avoided by using this endotracheal tube with a flat mouth and side opening. When intubation is assisted by using a bronchoscope or bougie, choosing an endotracheal tube with a flat mouth, a side opening and a suitable diameter can make tube delivery even easier. As a result, there will be a significant increase in the success rate of intubation. In this invention, the technical scheme adopted for solving the problem can be summarized as follows: As far as the parts of the endotracheal tube are concerned, there is difference when compared to the ordinary endotracheal tube in use today. The only difference is that this endotracheal tube is designed to be equipped with a circularly shaped flat mouth at the front. Its inner diameter is 5-10 mm. In addition, it has a side opening, which is elliptical in shape to enhance ventilation. There is also a matching guide core. This has a hemisphere at the front with a diameter of 3-5 mm. It is followed by a front cylinder, which is 30-50 mm in length, its diameter being equal to the diameter of the hemisphere in front. The front cylinder is followed by a truncated cone. The diameter of the front cross section of the truncated cone is equal to the diameter of the front cylinder. The diameter at the rear cross section of the truncated cone is equal to the diameter of the rear cylinder. The length of the truncated cone is 10-30 mm. It is followed by a rear cylinder with a diameter of 5-9 mm and a length of 50-70 cm.

The advantages of this invention can be summarized as follows. There is minimal contact between the endotracheal tube and the larynx. Relatively smaller components, such as the leading hemisphere and front cylinder, pass through the glottis into the trachea first. Because of this the field of view of the physician performing intubation is not blocked by the endotracheal tube. The small components are followed by insertion of gradually thickened components such as the truncated cone, rear cylinder and endotracheal tube with a flat mouth and side opening, which are introduced into the trachea through the glottis. There will be a significant increase in the success rate of intubation. Damage done to the vocal cords, epiglottis and piriform sinus by an endotracheal tube can be avoided. In comparison to an ordinary endotracheal tube with a slanted mouth, unless the slanted mouth is directly aligned with the glottis fissure, damage to the vocal cords and larynx can occur extremely easily when brute force is used by the physician performing intubation if obstruction is encountered during intubation. In addition, intubation is made more difficult with existing endotracheal tubes.

### 4. DESCRIPTION OF ATTACHED DRAWING

The following is a more detailed description of this invention in conjunction with the drawing and preferred embodiments.

FIGS. 1 and 2 are schematic diagrams of this invention.

FIG. 1 Endotracheal Tube with Flat Mouth and Side Opening, where 1—flat mouth; 2—side opening; 3—air-pocket cuff; 4—inner diameter (5.5-10 mm); 5—tube body.

FIG. 2 Guide Core, where 1—hemisphere (3—5 mm in diameter); 2—front cylinder (30-50 mm in length and diameter equal to the diameter of the hemisphere in front); 3—truncated cone (diameter of the front cross section is equal to the diameter of the front cylinder, while the diameter of the rear cross section is equal to the diameter of the rear cylinder, and its length is equal to 10-30 mm); 4—rear cylinder (5-9 mm in diameter and 50-70 cm in length).

### 5. PREFERRED EMBODIMENT

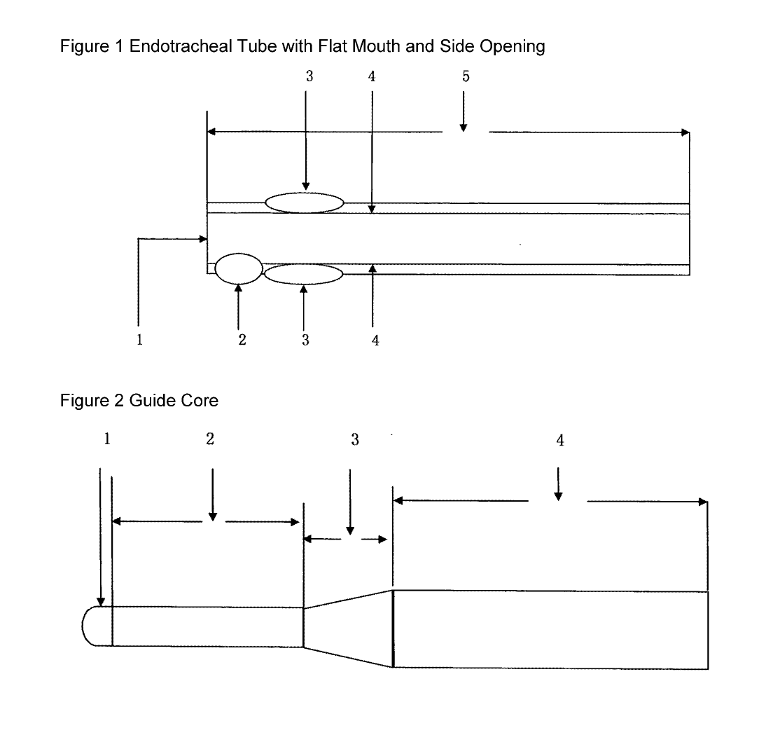

FIG. 1 shows an endotracheal tube with a flat mouth and a side opening. The front end of the endotracheal tube has a circularly shaped flat mouth. There is a side opening next to the flat mouth. There is an air-pocket cuff after the opening, which can be filled with air. The inner diameter of the flat mouth is 5-10 mm. FIG. 2 shows the guide core. The front end is a hemisphere with a diameter of 3-5 mm. It is followed by a front cylinder with a length of 30-50 mm and a diameter equal to the diameter of the hemisphere in front. The front cylinder is followed by a truncated cone. The diameter of the front cross section of the truncated cone is equal to the diameter of the front cylinder and the diameter of its rear cross section is equal to the diameter of the rear cylinder, and the length of the truncated cone is equal to 10-30 mm. It is followed by a rear cylinder with a diameter equal to 5-9 mm and a length of 50-70 cm.

The inner diameter of this flat mouthed endotracheal tube with a side opening is 5-10 mm. An endotracheal tube with a suitable inner diameter can be chosen by the anesthesiologist, based on the patient's requirement. The diameter of the rear cylinder of the guide core is 1-2 mm smaller than the inner diameter of the endotracheal tube. In patients for whom difficulties are anticipated in intubation, the guide core may be inserted into the trachea first, by introducing the front hemisphere, front cylinder, truncated cone and rear cylinder through the glottis. Then, the flat-mouthed endotracheal tube with a side opening can be inserted along the guide core. The guide core can then be withdrawn. Alternatively, the guide core may be inserted into the flat-mouthed endotracheal tube having a side opening up to the junction of the rear cylinder and the truncated cone. The physician performing intubation may hold the junction between the end of the endotracheal tube and its guide core to allow the leading hemisphere, front cylinder, truncated cone and rear cylinder to pass through the glottis in that particular order. After the air-pocket cuff enters the glottis, the guide core is withdrawn gradually. After the endotracheal tube is inserted to the desired depth, the guide core can be totally withdrawn and the air-pocket cuff may be inflated. After confirming that the endotracheal tube is located in the trachea, intubation is completed.

1. An endotracheal tube with a flat mouth and a side opening, equipped with a circularly shaped flat mouth, a side opening and an air-pocket cuff; and a matching guide core comprising a hemisphere, a front cylinder, a truncated cone and a rear cylinder.

2. The endotracheal tube with a flat mouth and a side opening as described in claim 1, which is further characterized by a circularly shaped flat mouth at the front, a side opening and an air-pocket cuff, and has an inner diameter of 5-10 mm.

3. The guide core as described in claim 1, comprising a hemisphere, a front cylinder, a truncated cone and a rear cylinder, and further characterized as follows: the front hemisphere is 3-5 mm in diameter; the front cylinder is 30-50 mm in length and its diameter is equal to the diameter of the leading hemisphere; the truncated cone is 10-30 mm in length and the diameter of its front cross section is equal to the diameter of the front cylinder, while the diameter of its rear cross section is equal to the diameter of the rear cylinder; and the rear cylinder is 5-9 mm in diameter and 50-70 cm in length.
